(19) United States
(12) Patent Application Publication
(10) Pub. No.: US 2021/0189341 A1
(43) Pub. Date: Jun. 24, 2021

(54) PLATELET DIAGNOSTIC IMAGING AGENTS

(71) Applicant: Cellphire, Inc., Rockville, MD (US)

(72) Inventors: Daniel Allen Sheik, Rockville, MD (US); Keith Andrew Moskowitz, Westfield, IN (US)

(21) Appl. No.: 17/104,548

(22) Filed: Nov. 25, 2020

Related U.S. Application Data
(60) Provisional application No. 62/941,508, filed on Nov. 27, 2019.

(51) Int. Cl. C12N 5/078; A61K 35/19; A61K 49/18; A01N 1/02

(52) U.S. Cl. CPC C12N 5/0644; A61K 35/19; A61K 49/1887; A01N 1/0221; A61K 49/1857

(57) ABSTRACT

Provided herein are MRI agent-loaded platelets, methods of preparing MRI agent-loaded platelets, and methods of using MRI agent-loaded platelets. In some embodiments, methods of loading MRI agents into platelets include contacting platelets with an MRI agent, a cell penetrating peptide, and a loading buffer that can include a salt, a base, a loading agent, and optionally at least one organic solvent.

### CROSS-REFERENCE TO RELATED APPLICATIONS

This application claims priority to U.S. Provisional Patent Application No. 62/941,508, filed on Nov. 27, 2019, the contents of which are incorporated herein by reference in its entirety.

### TECHNICAL FIELD

Provided herein are compositions and methods for use of platelets, platelet derivatives, or thrombosomes (e.g., freeze-dried platelet derivatives) as biological carriers of cargo, such as MRI agents, also referred to herein as MRI agent-loaded platelets, platelet derivatives, or thrombosomes. Also provided herein are methods of preparing platelets, platelet derivatives, or thrombosomes loaded with the MRI agent of interest.

MRI agent-loaded platelets described herein can be stored under typical ambient conditions, refrigerated, cryopreserved, for example with dimethyl sulfoxide (DMSO), and/or lyophilized after stabilization (e.g., to form thrombosomes)

### BACKGROUND

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Unactivated platelets, which are also commonly referred to as thrombocytes, are small, often irregularly-shaped (e.g., discoidal or ovoidal) megakaryocyte-derived components of blood that are involved in the clotting process. They aid in protecting the body from excessive blood loss due not only to trauma or injury, but to normal physiological activity as well. Platelets are considered crucial in normal hemostasis, providing the first line of defense against blood escaping from injured blood vessels. Platelets generally function by adhering to the lining of broken blood vessels, in the process becoming activated, changing to an amorphous shape, and interacting with components of the clotting system that are present in plasma or are released by the platelets themselves or other components of the blood. Purified platelets have found use in treating subjects with low platelet count (thrombocytopenia) and abnormal platelet function (thrombasthenia). Concentrated platelets are often used to control bleeding after injury or during acquired platelet function defects or deficiencies, for example those occurring during surgery and those due to the presence of platelet inhibitors.

Loading platelets with magnetic resonance imaging (MRI) agents may allow targeted delivery of the MRI agents to sites of interest. MRI agents can be used to image blood vessels and inflamed or diseased tissue where blood vessels have become compromised (e.g., “leaky”). Coupled peptide nucleic acids (PNA) including gadolinium-based MRI agents have been delivered intracellulary using cell-penetrating peptides (Mishra, R., et. al., Cell-Penetrating Peptides and Peptide Nucleic Acid-Coupled MRI Contrast Agents: Evaluation of Cellular Delivery and Target Binding, Bioconjugate Chem. 20, 1860-1868 (2009))

Further, MRI agent-loaded platelets may be lyophilized or cryopreserved to allow for long-term storage. In some embodiments, the loading of an MRI agent in the platelets can mitigate systemic side effects associated with the MRI agent and can shield the MRI agent from natural clearance mechanisms during migration to the site of injury. In some embodiments, the accumulation of MRI agent loaded platelets at the site of injury can enhance the resolution of magnetic resonance images and allow for improved disease diagnoses.

### SUMMARY OF THE INVENTION

Provided herein are methods of preparing MRI agent-loaded platelets, MRI agent-loaded platelet derivatives, or MRI agent-loaded thrombosomes (e.g., freeze-dried platelet derivatives), comprising: contacting platelets, platelet derivatives, or thrombosomes with an MRI agent, and at least one loading agent and optionally one or more plasticizers such as organic solvents, such as organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, to form the MRI agent-loaded platelets, MRI agent-loaded platelet derivatives, or MRI agent-loaded thrombosomes.

In some embodiments, the methods of preparing MRI agent-loaded platelets can include contacting the platelets, the platelet derivatives, and/or the thrombosomes with the MRI agent and with one loading agent. In some embodiments, the methods of preparing MRI agent-loaded platelets, MRI agent-loaded platelet derivatives, or MRI agent-loaded thrombosomes can include contacting the platelets, the platelet derivatives, or the thrombosomes with the MRI agent and with multiple loading agents.

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents include, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. The presence of organic solvents, such as ethanol, can be beneficial in the processing of platelets, platelet derivatives, and/or thrombosomes. In some embodiments, the organic solvent may open up and/or increase the flexibility of the plasma membrane of the platelets, platelet derivatives, and/or thrombosomes.

In some embodiments, provided herein is a method of preparing MRI agent-loaded platelets, MRI agent-loaded platelet derivatives, or MRI agent-loaded thrombosomes, comprising: contacting platelets, platelet derivatives, or thrombosomes with a MRI agent, and a loading buffer comprising a base, a loading agent, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, to form the MRI agent-loaded platelets, the MRI agent-loaded platelet derivatives, or the MRI agent-loaded thrombosomes.

In some embodiments, provided herein is a method of preparing MRI agent-loaded platelets, MRI agent-loaded platelet derivatives, or MRI agent-loaded thrombosomes, comprising: contacting platelets, platelet derivatives, or thrombosomes with a MRI agent and a loading buffer comprising a salt, a base, a loading agent, and optionally at least one organic solvent to form the MRI agent-loaded platelets, MRI agent-loaded platelet derivatives, or the MRI agent-loaded thrombosomes.

In some embodiments, provided herein is a method of preparing MRI-loaded platelets, MRI-loaded platelet derivatives, or MRI-loaded thrombosomes, comprising: contacting platelets, platelet derivatives, or thrombosomes with an MRI and with a loading agent and optionally at least one organic solvent to form the MRI-loaded platelets, the MRI-loaded platelet derivatives, or the MRI-loaded thrombosomes.

In some embodiments, provided herein is a method of preparing MRI agent-loaded platelets, including: contacting platelets with an MRI agent coupled to a cell penetrating peptide; and a loading buffer comprising a salt, a base, a loading agent, and optionally at least one organic solvent, to form the MRI agent-loaded platelets.

In some embodiments, provided herein is a method of preparing MRI agent-loaded platelets, including: a) providing platelets; and b) contacting the platelets with an MRI agent coupled to a cell penetrating peptide; and a loading buffer comprising a salt, a base, a loading agent, and optionally at least one organic solvent to form the MRI agent-loaded platelets.

In some embodiments, provided herein is a method where the platelets are contacted with the MRI agent coupled to a cell penetrating peptide and with the loading buffer sequentially, in either order.

In some embodiments, provided herein is a method of preparing MRI agent-loaded platelets, including: (1) contacting the platelets with a loading buffer comprising a salt, a base, a loading agent, and optionally at least one organic solvent to form a first composition; and (2) contacting the first composition with an MRI agent coupled to a cell penetrating peptide, to form the MRI agent-loaded platelets.

In some embodiments, provided herein is a method where the platelets are contacted with the MRI agent coupled to the cell penetrating peptide and with the loading buffer concurrently.

In some embodiments, provided herein is method of preparing MRI agent-loaded platelets, including: contacting the platelets with an MRI agent in the presence of a cell penetrating peptide and a loading buffer comprising a salt, a base, a loading agent, and optionally at least one organic solvent to form the MRI agent-loaded platelets.

In some embodiments, provided herein is a method where the platelets are pooled from a plurality of donors.

In some embodiments, provided herein is a method of preparing MRI agent-loaded platelets including: A) pooling platelets from a plurality of donors; and B) contacting the platelets from (A) with an MRI agent coupled to a cell penetrating peptide; and with a loading buffer comprising a salt, a base, a loading agent, and optionally at least one organic solvent, to form the MRI agent-loaded platelets.

In some embodiments, provided herein is a method of preparing MRI agent-loaded including: A) pooling platelets from a plurality of donors; and B) (1) contacting the platelets from (A) with an MRI agent coupled to a cell penetrating peptide to form a first composition; and (2) contacting the first composition with a loading buffer comprising a salt, a base, a loading agent, and optionally at least one organic solvent, to form the MRI agent-loaded platelets.

In some embodiments, provided herein is a method of preparing MRI agent-loaded platelets including: A) pooling platelets from a plurality of donors; and B) (1) contacting the platelets from (A) with a loading buffer comprising a salt, a base, a loading agent, and optionally at least one organic solvent, to form a first composition; and (2) contacting the first composition with an MRI agent coupled to a cell penetrating peptide to form the MRI agent-loaded platelets.

In some embodiments, provided herein is a method of preparing MRI agent-loaded platelets including: A) pooling platelets from a plurality of donors; and B) contacting the platelets with an MRI agent coupled to a cell penetrating peptide and a loading buffer comprising a salt, a base, a loading agent, and optionally at least one organic solvent, to form the MRI agent-loaded platelets.

In some embodiments, provided herein is a method where the loading buffer comprises optionally at least one organic solvent.

In some embodiments, provided herein is a method where the loading agent is a monosaccharide or a disaccharide.

In some embodiments, provided herein, is a method where the loading agent is sucrose, maltose, dextrose, trehalose, glucose, mannose, or xylose.

In some embodiments, provided herein is a method where the platelets are isolated prior to a contacting step.

In some embodiments, provided herein is a method where the platelets are selected from the group consisting of fresh platelets, stored platelet, and any combination thereof.

In some embodiments, provided herein is a method where the MRI agent comprises Gadolinium.

In some embodiments, provided herein is a method where the MRI agent comprises a nanoparticle.

In some embodiments, provided herein is a method where the cell penetrating peptide is Tat, or a portion thereof.

In some embodiments, provided herein is a method where the platelets are loaded with the MRI agent in a period of time of 1 minute to 48 hours.

In some embodiments, provided herein is a method where the concentration of MRI agent in the MRI agent-loaded platelets is from about 0.1 nM to about 10 μM.

In some embodiments, provided herein is a method where the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

In some embodiments, provided herein is a method further including cold storing, cryopreserving, freeze-drying, thawing, rehydrating, and combinations thereof the MRI agent-loaded platelets.

In some embodiments, provided herein is a method where the drying step comprises freeze-drying the MRI agent-loaded platelets.

In some embodiments, provided herein is a method further including rehydrating the MRI agent-loaded platelets obtained from the drying step.

Also provided herein are MRI agent-loaded platelets prepared by any of the preceding methods.

Also provided herein are MRI agent-loaded platelets prepared by a method including rehydrating the MRI agent-loaded platelets.

In some embodiments, provided herein is a method where the method does not comprise contacting the platelets with an organic solvent.

In some embodiments, provided herein is a method, where the method does not comprise contacting the first composition with an organic solvent.

In some embodiments, provided herein is a method where the method includes contacting the platelets with Prostaglandin E1.

In some embodiments, provided herein is a method were the method does not include contacting the platelets with Prostaglandin E1.

In some embodiments, provided herein is a method, where the method includes contacting the plates with GR144053.

In some embodiments, provided herein is a method where the method does not include contacting the platelets with GR144053.

In some embodiments, provided herein is a method where the method includes contacting the platelets with eptifibatide.

In some embodiments, provided herein is a method where the method does not include contacting the platelets with eptifibatide.

Thus, in some embodiments provided herein is a method of preparing MRI-loaded platelets, the MRI-loaded platelet derivatives, or the MRI-loaded thrombosomes, comprising: contacting the platelets, the platelet derivatives, or the thrombosomes with a MRI in the presence of a buffer comprising a salt, a base, a loading agent, and optionally ethanol, to form the MRI-loaded platelets, the MRI-loaded platelet derivatives, or the MRI-loaded thrombosomes.

In some embodiments, the methods further include drying the MRI-loaded platelets or the MRI-loaded platelet derivatives. In some embodiments, the methods further include freeze-drying the MRI-loaded platelets or the MRI-loaded platelet derivatives. In such embodiments, the methods further include rehydrating the MRI-loaded platelets or the MRI-loaded platelet derivatives obtained from the drying step.

In some embodiments, the methods that further include drying the MRI-loaded platelets or the MRI-loaded platelet derivatives and rehydrating the MRI-loaded platelets or the MRI-loaded platelet derivatives obtained from the drying step provides rehydrated platelets or the thrombosomes comprising at least 10% of the amount of the MRI prior to loading.

In some embodiments of the methods of preparing cargo-loaded platelets, such as MRI agent-loaded platelets, as provided herein, the methods do not comprise contacting platelets, platelet derivatives, or thrombosomes with ethanol.

In some embodiments of the methods of preparing cargo-loaded platelets, such as MRI agent-loaded platelets, as provided herein, the methods do not comprise contacting platelets, platelet derivatives, or thrombosomes with a solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

In some embodiments of the methods of preparing cargo-loaded platelets, such as MRI agent-loaded platelets, as provided herein, the methods do not comprise contacting platelets, platelet derivatives, or thrombosomes with an organic solvent.

In some embodiments of the methods of preparing cargo-loaded platelets, such as MRI agent-loaded platelets, as provided herein, the methods do not comprise contacting platelets, platelet derivatives, or thrombosomes with a solvent.

In some embodiments of the methods of preparing cargo-loaded platelets, such as MRI agent-loaded platelets, as provided herein, the methods comprise contacting platelets, platelet derivatives, or thrombosomes with a solvent, such as an organic solvent, such as organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, such as ethanol.

In some embodiments, platelets, platelet derivatives, or thrombosomes are pooled from a plurality of donors. Such platelets, platelet derivatives, and thrombosomes pooled from a plurality of donors may be also referred herein to as pooled platelets, platelet derivatives, or thrombosomes. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35.

In some embodiments, the methods of preparing MRI agent-loaded platelets, MRI agent-loaded platelet derivatives, or MRI agent-loaded thrombosomes that include pooling platelets, platelet derivatives, or thrombosomes from a plurality of donors include a viral inactivation step.

In some embodiments, the methods of preparing MRI agent-loaded platelets, MRI agent-loaded platelet derivatives, or MRI agent-loaded thrombosomes that include pooling platelets, platelet derivatives, or thrombosomes from a plurality of donors do not include a viral inactivation step.

In some embodiments, the platelets, the platelet derivatives, or the thrombosomes are loaded with the MRI agent in a period of time of about less than 1 minute to 48 hours, such as 5 minutes to 24 hours, such as 20 minutes to 12 hours, such as 30 minutes to 6 hours, such as 1 hour to 3 hours, such as about 2 hours. In some embodiments, platelets, platelet derivatives, or thrombosomes are loaded with the MRI agent for a time of about 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or longer, or any time period range therein. In some embodiments, platelets, platelet derivatives, or thrombosomes are loaded with the MRI agent for a time of less than one minute. In some embodiments, a concentration of MRI agent from about 0.1 nM to about 10 μM, such as about 1 nM to about 1 μM, such as about 10 nM to 10 μM, such as about 100 nM is loaded in a period of time of about less than 1 minute to 48 hours, such as 5 minutes to 24 hours, such as 20 minutes to 12 hours, such as 30 minutes to 6 hours, such as 1 hour minutes to 3 hours, such as about 2 hours.

In some embodiments, provided herein are MRI agent-loaded platelets, MRI agent-loaded platelet derivatives, or MRI agent-loaded thrombosomes prepared according to any of the variety of methods disclosed herein. In some embodiments provided herein are rehydrated platelets, platelet derivatives, or thrombosomes prepared as according to any of the variety of methods disclosed herein.

In some embodiments, MRI agent-loaded platelets, MRI agent-loaded platelet derivatives, or MRI agent-loaded thrombosomes protect the MRI from metabolic degradation or inactivation. MRI delivery with MRI-loaded platelets, MRI-loaded platelet derivatives, or MRI-loaded thrombosomes may therefore be advantageous in diagnosing diseases such as cancer, since MRI-loaded platelets, MRI-loaded platelet derivatives, or MRI-loaded thrombosomes facilitate targeting of cancer cells while mitigating systemic side effects. MRI-loaded platelets, MRI-loaded platelet derivatives, or MRI-loaded thrombosomes may be used in any therapeutic setting in which expedited healing process is required or advantageous.

Accordingly, in some embodiments, provided herein is a method of enhancing diagnosis of a disease (e.g., any of the variety of diseases disclosed herein), comprising administering any of the variety of MRI agent-loaded platelets, MRI agent-loaded platelet derivatives, or MRI agent-loaded thrombosomes disclosed herein. Accordingly, in some embodiments, provided herein is a method of diagnosing a disease (e.g., any of the variety of diseases disclosed herein), comprising administering cold stored, room temperature stored, cryopreserved thawed, rehydrated, and/or lyophilized platelets, platelet derivatives, or thrombosomes as disclosed herein. In some embodiments, the disease is cancer. In some embodiments, the disease is Traumatic Brain injury. In some embodiments, the disease is stroke. In some embodiments, the disease is an embolism. In some embodiments, the disease is a hemorrhage.

### DESCRIPTION OF DRAWINGS

FIG. 1 shows pooled apheresis platelets incubated with FITC labeled TAT peptide in loading buffer.

FIGS. 2A-C shows platelets analyzed by flow cytometry for FITC-TAT loading in either HMTA or loading buffer at two concentrations (25 μM or 50 μM) by mean fluorescence intensity (FIG. 2A). FIGS. 2B and 2C show pooled apheresis platelets incubated FITC-labeled TAT peptide in either HMTA or loading buffer at either 50 μM FITC-labeled TAT (FIG. 2B) or 25 μM FITC-labeled TAT (FIG. 2C).

FIG. 3 is a flow cytometry histogram of samples incubated with 100 μM FITC-TAT in the presence of platelet anti-aggregation compounds PGE1, GR144053, and eptifibatide. PGE1 appears to be associated with improved platelet loading little to no effect is observed with GR144053 or eptifibatide on the distribution of FITC-CPP.

FIG. 4 shows the effect of different buffers on FITC-TAT loading into platelets as measured by fluorescence intensity.

FIGS. 5A-C shows brightfield, FITC, and overlaid microscopy images of non-loaded platelets (FIG. 5A), 100 μM fluorescein (FIG. 5B), and 100 μM FITC-labeled TAT (FIG. 5C).

FIG. 6 is a flow cytometry histogram of SAMPLS incubated with either loading buffer (left peak) or a solution of FITC-labeled magnetic nanoparticles (right peak).

FIGS. 7A-D shows Texas Red (FIG. 7A), FITC (FIG. 7B), brightfield (FIG. 7C), and overlaid (FIG. 7D) images of samples incubated with FITC-labeled magnetic nanoparticles.

FIG. 8 is a flow cytometry histogram of samples incubated with either loading buffer (left peak) or a 50 μM FITC-CPP-Gd-DOTA solution (right peak) for 30 minutes.

FIGS. 9A-B show a schematic (FIG. 9A) and magnetic resonance imaging (FIG. 9B). As shown in FIG. 9A, samples 1A, 1B, and 2B are negative controls, sample 2A includes Gd-DOTA-FITC-CPP with platelets (400 K/μL), samples indicated with 100 mM or 100 μM GdCl₃ are positive controls.

FIG. 10 is a graph showing post-cryopreservation occlusion time of platelets loaded with Gd-DOTA-FITC-CPP with plasma only (negative control), pooled, unloaded platelets (positive control), and Gd-DOTA-FITC-CPP loaded platelets.

### DETAILED DESCRIPTION

It is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the term “platelet” can include whole platelets, fragmented platelets, platelet derivatives, or thrombosomes. Thus, for example, reference to “MRI agent-loaded platelets” may be inclusive of MRI agent-loaded platelets as well as MRI agent-loaded platelet derivatives or MRI agent-loaded thrombosomes, unless the context clearly dictates a particular form.

As used herein, “thrombosomes” (sometimes also herein called “Tsomes” or “Ts”, particularly in the Examples and Figures) are platelet derivatives that have been contacted with an incubating agent (e.g., any of the incubating agents described herein) and lyopreserved (e.g., freeze-dried). In some cases, thrombosomes can be prepared from pooled platelets. Thrombosomes can have a shelf life of 2-3 years in dry form at ambient temperature and can be rehydrated with sterile water within minutes for immediate infusion.

As used herein and in the appended claims, the term “fresh platelet” can include day of use platelets.

As used herein and in the appended claims the term “stored platelet” can include platelets stored for approximately 24 hours or longer before use.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

In some embodiments, rehydrating the MRI agent-loaded platelets includes adding to the platelets an aqueous liquid. In some embodiments, the aqueous liquid is water. In some embodiments, the aqueous liquid is an aqueous solution. In some embodiments, the aqueous liquid is a saline solution. In some embodiments, the aqueous liquid is a suspension.

In some embodiments, the rehydrated platelets have coagulation factor levels showing all individual factors (e.g., Factors VII, VIII and IX) associated with blood clotting at 40 international units (IU) or greater.

In some embodiments, the dried platelets, such as freeze-dried platelets, have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets, have less than about 10%, such as less than about 8%, such as less than about 6%, such as less than about 4%, such as less than about 2%, such as less than about 0.5% crosslinking of platelet membranes via proteins and/or lipids present on the membranes.

In some embodiments, the MRI agent-loaded platelets and the dried platelets, such as freeze-dried platelets, having a particle size (e.g., diameter, max dimension) of at least about 0.2 μm (e.g., at least about 0.3 μm, at least about 0.4 μm, at least about 0.5 μm, at least about 0.6 μm, at least about 0.7 μm, at least about 0.8 μm, at least about 0.9 μm, at least about 1.0 μm, at least about 1.0 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets and/or the dried platelets, such as freeze-dried platelets, have a particle size in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of platelets and/or the dried platelets, such as freeze-dried platelets, are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of platelets and/or the dried platelets, such as freeze-dried platelets, are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, platelets are isolated prior to treating (e.g., contacting) the platelets with an MRI agent.

Accordingly, in some embodiments, the methods for preparing an MRI agent-loaded platelets includes: step (a) isolating platelets, for example in a liquid medium; and step (b) treating the platelets with an MRI agent coupled to a cell penetrating peptide and with a loading buffer comprising a salt, a base, a loading agent, and optionally ethanol, to form the MRI agent-loaded platelets.

Accordingly, in some embodiments, the methods for preparing an MRI agent-loaded platelets includes: step (a) isolating platelets, for example in a liquid medium; and step (b) contacting the platelets with an MRI agent coupled to a cell penetrating peptide and with a loading buffer comprising a salt, a base, a loading agent, and optionally ethanol, to form the MRI agent-loaded platelets.

Accordingly, in some embodiments, the methods for preparing MRI agent-loaded platelets includes: step (a) isolating platelets, for example in a liquid medium; step (b) treating the platelets with an MRI agent coupled with a cell penetrating peptide to form a first composition; and step (c) treating the first composition with a buffer comprising a salt, a base, a loading agent, and optionally at least one organic solvent to form the MRI agent-loaded platelets.

Accordingly, in some embodiments, the methods for preparing MRI agent-loaded platelets includes: step (a) isolating platelets, for example in a liquid medium; step (b) contacting the platelets with an MRI agent coupled with a cell penetrating peptide to form a first composition; and step (c) contacting the first composition with a buffer comprising a salt, a base, a loading agent, and optionally at least one organic solvent to form the MRI agent-loaded platelets.

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof.

Accordingly, in some embodiments, the methods for preparing MRI agent-loaded platelets includes: step (a) isolating platelets, for example in a liquid medium; step (b) treating the platelets with a buffer comprising a salt, a base, a loading agent, and optionally at least one organic solvent, to form a first composition; and step (c) treating the first composition with an MRI agent coupled to a cell penetrating peptide, to form the MRI agent-loaded platelets.

Accordingly, in some embodiments, the methods for preparing MRI agent-loaded platelets includes: step (a) isolating platelets, for example in a liquid medium; step (b) contacting the platelets with a buffer comprising a salt, a base, a loading agent, and optionally at least one organic solvent, to form a first composition; and step (c) contacting the first composition with an MRI agent coupled to a cell penetrating peptide, to form the MRI agent-loaded platelets.

In some embodiments, isolating platelets includes isolating platelets from blood.

In some embodiments, platelets are donor-derived platelets. In some embodiments, platelets are obtained by a process that includes an apheresis step. In some embodiments, platelets are fresh platelets. In some embodiments, platelets are stored platelets.

In some embodiments, platelets are derived in vitro. In some embodiments, platelets are derived or prepared in a culture prior to treating the platelets with an MRI agent. In some embodiments, preparing the platelets includes deriving or growing the platelets from a culture of megakaryocytes. In some embodiments, preparing the platelets includes deriving or growing the platelets (or megakaryocytes) from a culture of human pluripotent stem cells (PCSs), including embryonic stem cells (ESCs) and/or induced pluripotent stem cells (iPSCs).

Accordingly, in some embodiments, the methods for preparing MRI agent-loaded platelets includes: step (a) providing platelets; and step (b) treating the platelets with an MRI agent reagent, and with a loading buffer comprising a salt, a base, a loading agent, and optionally at least one organic solvent, to form the MRI agent-loaded platelets.

Accordingly, in some embodiments, the methods for preparing MRI agent-loaded platelets includes: step (a) providing platelets; and step (b) contacting the platelets with an MRI agent reagent, and with a loading buffer comprising a salt, a base, a loading agent, and optionally at least one organic solvent, to form the MRI agent-loaded platelets.

Accordingly, in some embodiments, the methods for preparing MRI agent-loaded platelets includes: step (a) providing platelets; step (b) treating the platelets with an MRI agent to form a first composition; and step (c) treating the first composition with a buffer comprising a salt, a base, a loading agent, and optionally at least one organic solvent, to form the MRI agent-loaded platelets.

Accordingly, in some embodiments, the methods for preparing MRI agent-loaded platelets includes: step (a) providing platelets; step (b) contacting the platelets with an MRI agent to form a first composition; and step (c) contacting the first composition with a buffer comprising a salt, a base, a loading agent, and optionally at least one organic solvent, to form the MRI agent-loaded platelets.

Accordingly, in some embodiments, the methods for preparing MRI agent-loaded platelets includes: step (a) providing platelets; step (b) treating the platelets with a buffer comprising a salt, a base, a loading agent, and optionally at least one organic solvent, to form a first composition; and step (c) treating the first composition with an MRI agent, to form the MRI agent-loaded platelets.

Accordingly, in some embodiments, the methods for preparing MRI agent-loaded platelets includes: step (a) providing platelets; step (b) contacting the platelets with a buffer comprising a salt, a base, a loading agent, and optionally at least one organic solvent, to form a first composition; and step (c) contacting the first composition with an MRI agent, to form the MRI agent-loaded platelets.

In some embodiments, no solvent is used. Thus, in some embodiments, the method for preparing MRI agent-loaded platelets comprises:
a) isolating platelets, for example in a liquid medium; and
b) treating the platelets with an MRI agent and with a loading buffer comprising a salt, a base, and a loading agent, to form the MRI agent-loaded platelets,
wherein the method does not comprise treating the platelets with an organic solvent such as ethanol.

In some embodiments, no solvent is used. Thus, in some embodiments, the method for preparing MRI agent-loaded platelets comprises:
a) isolating platelets, for example in a liquid medium;
   and
b) contacting the platelets with an MRI agent and with a loading buffer comprising a salt, a base, and a loading agent, to form the MRI agent-loaded platelets, wherein the method does not comprise contacting the platelets with an organic solvent such as ethanol.

Thus, in some embodiments, the method for preparing MRI agent-loaded platelets comprises:
a) isolating platelets, for example in a liquid medium;
b) treating the platelets with an MRI agent to form a first composition; and
c) treating the first composition with a buffer comprising a salt, a base, and a loading agent, to form the MRI agent-loaded platelets,
wherein the method does not comprise treating the platelets with an organic solvent such as ethanol and the method does not comprise treating the first composition with an organic solvent such as ethanol.

Thus, in some embodiments, the method for preparing MRI agent-loaded platelets comprises:
a) isolating platelets, for example in a liquid medium;
b) contacting the platelets with an MRI agent to form a first composition; and
c) contacting the first composition with a buffer comprising a salt, a base, and a loading agent, to form the MRI agent-loaded platelets, wherein the method does not comprise contacting the platelets with an organic solvent such as ethanol and the method does not comprise treating the first composition with an organic solvent such as ethanol.

Thus, in some embodiments, the method for preparing MRI agent-loaded platelets comprises:
a) isolating platelets, for example in a liquid medium;
b) treating the platelets with a buffer comprising a salt, a base, and a loading agent, to form a first composition; and
c) treating the first composition with an MRI agent, to form the MRI agent-loaded platelets.
   wherein the method does not comprise treating the platelets with an organic solvent such as ethanol and the method does not comprise treating the first composition with an organic solvent such as ethanol.

Thus, in some embodiments, the method for preparing MRI agent-loaded platelets comprises:
a) isolating platelets, for example in a liquid medium;
b) contacting the platelets with a buffer comprising a salt, a base, and a loading agent, to form a first composition; and
c) contacting the first composition with an MRI agent, to form the MRI agent-loaded platelets.
   wherein the method does not comprise contacting the platelets with an organic solvent such as ethanol and the method does not comprise contacting the first composition with an organic solvent such as ethanol.

In some embodiments, the method for preparing MRI agent-loaded platelets comprises:
a) providing platelets; and
b) treating the platelets with an MRI agent-loaded and with a loading buffer comprising a salt, a base, and a loading agent, to form the MRI agent-loaded platelets,
wherein the method does not comprise treating the platelets with an organic solvent such as ethanol.

In some embodiments, the method for preparing MRI agent-loaded platelets comprises:
a) providing platelets;
   and
b) contacting the platelets with an MRI agent-loaded and with a loading buffer comprising a salt, a base, and a loading agent, to form the MRI agent-loaded platelets,
   wherein the method does not comprise contacting the platelets with an organic solvent such as ethanol.

Thus, in some embodiments, the method for preparing MRI agent-loaded platelets comprises:

a) providing platelets;

b) treating the platelets with an MRI agent to form a first composition; and

c) treating the first composition with a buffer comprising a salt, a base, and a loading agent, to form the MRI agent-loaded platelets,

wherein the method does not comprise treating the platelets with an organic solvent such as ethanol and the method does not comprise treating the first composition with an organic solvent such as ethanol.

Thus, in some embodiments, the method for preparing MRI agent-loaded platelets comprises:
a) providing platelets;
b) contacting the platelets with an MRI agent to form a first composition; and
   c) contacting the first composition with a buffer comprising a salt, a base, and a loading agent, to form the MRI agent-loaded platelets,
   wherein the method does not comprise contacting the platelets with an organic solvent such as ethanol and the method does not comprise contacting the first composition with an organic solvent such as ethanol.

Thus, in some embodiments, the method for preparing MRI agent-loaded platelets comprises:
a) providing platelets;
b) treating the platelets with a buffer comprising a salt, a base, and a loading agent, to form a first composition; and
c) treating the first composition with an MRI agent, to form the MRI agent-loaded platelets. wherein the method does not comprise treating the platelets with an organic solvent such as ethanol and the method does not comprise treating the first composition with an organic solvent such as ethanol.

Thus, in some embodiments, the method for preparing MRI agent-loaded platelets comprises:
a) providing platelets;
b) contacting the platelets with a buffer comprising a salt, a base, and a loading agent, to form a first composition; and
c) contacting the first composition with an MRI agent, to form the MRI agent-loaded platelets, wherein the method does not comprise contacting the platelets with an organic solvent such as ethanol and the method does not comprise contacting the first composition with an organic solvent such as ethanol.

In some embodiments, the loading agent is a saccharide. In some embodiments, the saccharide is a monosaccharide. In some embodiments, the saccharide is a disaccharide. In some embodiments, the saccharide is a non-reducing disaccharide. In some embodiments, the saccharide is sucrose, maltose, trehalose, glucose (e.g., dextrose), mannose, or xylose. In some embodiments, the loading agent is a starch.

As used herein, the term “MRI agent” is any agent that is suitable for magnetic resonance imaging described herein or known in the art.

In some embodiments, an MRI agent loaded into platelets is modified. For example, an MRI agent can be modified to increase its stability during the platelet loading process, while the MRI agent is loaded into the platelet, and/or after the MRI agent's release from a platelet. In some embodiments, the modified MRI agent's stability is increased with little or no adverse effect on its activity. For example, the modified MRI agent can have at least 70%, 75%, 80%, 85%, 90%, 95%, 96%, 97%, 98%, 99% or more of the activity of the corresponding unmodified MRI agent. In some embodiments, the modified MRI agent has 100% (or more) of the activity of the corresponding unmodified MRI agent. Various modifications that stabilize MRI agents are known in the art. In some embodiments, the MRI agent is stabilized by one or more of a stabilizing oligonucleotide (see, e.g., U.S. Application Publication No. 2018/0311176), a backbone/side chain modification (e.g., a 2-sugar modification such as a 2′-fluor, methoxy, or amine substitution, or a 2′-thio (—SH), 2′-azido (—N3), or 2′-hydroxymethyl (—CH2OH) modification), an unnatural nucleic acid substitution (e.g., an S-glycerol, cyclohexenyl, and/or threose nucleic acid substitution, an L-nucleic acid substitution, a locked nucleic acid (LNA) modification (e.g., the ribose moiety of an LNA nucleotide is modified with an extra bridge connecting the 2′ oxygen and 4′ carbon), conjugation with PEG, a nucleic acid bond modification or replacement (e.g., a phosphorothioate bond, a methylphosphonate bond, or a phosphorodiamidate bond), a reagent or reagents (e.g., intercalating agents such as coralyne, neomycin, and ellipticine; also see US Publication Application Nos. 2018/0312903 and 2017/0198335, each of which are incorporated herein by reference in their entireties, for further examples of stabilizing reagents).

In some embodiments, an MRI agent loaded into platelets is modified to include an imaging agent. For example, an MRI agent can be modified with an imaging agent in order to image the MRI agent loaded platelet in vivo. In some embodiments, an MRI agent can be modified with two or more imaging agents (e.g., any two or more of the imaging agents described herein). In some embodiments, an MRI agent loaded into platelets is modified with a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as ⁵⁴Cu, ⁴⁸V, ⁵²Fe, ⁵⁵Co, ⁹⁴Tc or ⁶⁸Ga; or gamma-emitters such as ¹⁷¹Tc, ¹¹¹In, ¹¹³In, or ⁶⁷Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to 123I, ¹³¹I or ⁷⁷Br. For example, a positron-emitting radioactive non-metal can include, but is not limited to ¹¹C, ¹³N, ¹⁵O, ¹⁷F, ⁷⁵Br, ⁷⁶Br or ¹²⁴I. For example, a hyperpolarized NMR-active nucleus can include, but is not limited to ¹³C, ¹⁵N, ¹⁹F, ²⁹Si and ³¹P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s(dithiolene) complexes, bₗₛ(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals ⁶⁷Cu, ⁸⁹Sr, ⁹⁰Y, ¹⁵³Sm, ¹⁸⁵Re, ¹⁸⁸Re or ¹⁹²Ir, and non-metals ³²P, ³³P, ³⁸S, ³⁸Cl, ³⁹Cl, ⁸²Br and ⁸³Br. In some embodiments, an MRI agent loaded into platelets can be associated with gold or other equivalent metal particles (such as nanoparticles). For example, a metal particle system can include, but is not limited to gold nanoparticles (e.g., Nanogold™).

In some embodiments, an MRI agent loaded into platelets that is modified with an imaging agent is imaged using an imaging unit. The imaging unit can be configured to image the MRI agent loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Chen, Z., et. al., Advance of Molecular Imaging Technology and Targeted Imaging Agent in Imaging and Therapy, Biomed Res Int., 819324, doi: 10.1155/2014/819324 (2014) have described various imaging techniques and which is incorporated by reference herein in its entirety.

In some embodiments, such as embodiments wherein the platelets are treated (e.g., contacted) with the MRI agent and the loading buffer sequentially as disclosed herein, the MRI agent may be loaded in a liquid medium that may be modified to change the proportion of media components or to exchange components for similar products, or to add components necessary for a given application.

In some embodiments, the loading buffer and/or the liquid medium include one or more of a) water or a saline solution, b) one or more additional salts, or c) a base. In some embodiments, the loading buffer, and/or the liquid medium, may include one or more of a) DMSO, b) one or more salts, or d) a base.

In some embodiments, the loading agent is loaded into the platelets in the presence of an aqueous medium. In some embodiments, the loading agent is loaded in the presence of a medium comprising DMSO. As an example, one embodiment of the methods herein includes treating (e.g., contacting) platelets with an MRI agent coupled with a cell penetrating peptide and with an aqueous loading buffer comprising a salt, a base, a loading agent, and optionally at least one organic solvent, to form the MRI agent-loaded platelets. As an example, one embodiment of the methods herein includes treating (e.g., contacting) platelets with an MRI agent coupled with a cell penetrating peptide and with a loading buffer comprising DMSO and comprising a salt, a base, a loading agent, and optionally ethanol, to form the MRI agent-loaded platelets.

In some embodiments, the loading buffer and/or the liquid medium, include one or more salts selected from phosphate salts, sodium salts, potassium salts, calcium salts, magnesium salts, and any other salt that can be found in blood or blood products, or that is known to be useful in drying platelets, or any combination of two or more of these.

Preferably, these salts are present in the composition at an amount that is about the same as is found in whole blood.

In some embodiments, the MRI agent-loaded platelets are prepared by incubating the platelets with the MRI agent in the liquid medium for different durations at or at different temperatures from about 15-45° C., or about 22° C. In some embodiments, the MRI agent-loaded platelets are prepared by incubating the platelets with the MRI agent in the liquid medium at a temperature from about 18-42° C., about 20-40° C., about 22-37° C., or about 16° C., about 18° C., about 20° C., about 22° C., about 24° C., about 26° C., about 28° C., about 30° C., about 32° C., about 34° C., about 36° C., about 37° C., about 39° C., about 41° C., about 43° C., or about 45° C. for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the MRI agent-loaded platelets are prepared by incubating the platelets with the Mill agent in the liquid medium at a temperature from about 18-42° C., about 20-40° C., about 22-37° C., or about 16° C., about 18° C., about 20° C., about 22° C., about 24° C., about 26° C., about 28° C., about 30° C., about 32° C., about 34° C., about 36° C., about 37° C., about 39° C., about 41° C., about 43° C., or about 45° C. for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the MRI agent-loaded platelets are prepared by incubating the platelets with the MRI agent in the liquid medium from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs.

In some embodiments, the platelets are at a concentration from about 1,000 platelets/μl to about 10,000,000 platelets/μl. In some embodiments, the platelets are at a concentration from about 50,000 platelets/μl to about 4,000,000 platelets/μl. In some embodiments, the platelets are at a concentration from about 100,000 platelets/μl to about 300,000,000 platelets/μl. In some embodiments, the platelets are at a concentration from about 1,000,000 to about 2,000,000. In some embodiments, the platelets are at a concentration of about 200,000,000 platelets/μl.

In some embodiments, the MRI agent-loaded platelets are prepared by incubating the platelets with the MRI agent in the liquid medium for different durations. The step of incubating the platelets to load one or more MRI agent(s) includes incubating the platelets for a time suitable for loading, as long as the time, taken in conjunction with the temperature, is sufficient for the MRI agent to come into contact with the platelets and, preferably, be incorporated, at least to some extent, into the platelets. For example, in some embodiments, the MRI agent-loaded platelets are prepared by incubating the platelets with the MRI agent in the liquid medium for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the MRI agent-loaded platelets are prepared by incubating the platelets with the MRI agent in the liquid medium for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the MRI agent-loaded platelets are prepared by incubating the platelets with the MRI agent in the liquid medium from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs.

In some embodiments, the MRI agent-loaded platelets are prepared by incubating the platelets with the MRI agent in the liquid medium at different temperatures. The step of incubating the platelets to load one or more MRI agent(s), includes incubating the platelets with the MRI agent in the liquid medium at a temperature that, when selected in conjunction with the amount of time allotted for loading, is suitable for loading. In general, the platelets with the MRI agent in the liquid medium are incubated at a suitable temperature (e.g., a temperature above freezing) for at least a sufficient time for the MRI agent to come into contact with the platelets. In some embodiments, incubation is conducted at 22° C. In certain embodiments, incubation is performed at 4° C. to 45° C., such as 15° C. to 42° C. For example, in some embodiments, incubation is performed from about 18-42° C., about 20-40° C., about 22-37° C., or about 16° C., about 18° C., about 20° C., about 22° C., about 24° C., about 26° C., about 28° C., about 30° C., about 32° C., about 34° C., about 36° C., about 37° C., about 39° C., about 41° C., about 43° C., or about 45° C. for 110 to 130 (e.g., 120) minutes and for as long as 24-48 hours.

In some embodiments of the methods of preparing MRI agent-loaded platelets disclosed herein, the methods further include acidifying the platelets, or pooled platelets, to a pH of about 6.0 to about 7.4, prior to a treating (e.g., contacting) step disclosed herein. In some embodiments, the methods include acidifying the platelets to a pH of about 6.5 to about 6.9. In some embodiments, the methods include acidifying the platelets to a pH of about 6.6 to about 6.8. In some embodiments, the acidifying includes adding to the pooled platelets a solution comprising Acid Citrate Dextrose.

In some embodiments, the platelets are isolated prior to a treating (e.g., contacting) step. In some embodiments, the methods further include isolating platelets by using centrifugation. In some embodiments, the centrifugation occurs at a relative centrifugal force (RCF) of about 800 g to about 2000 g. In some embodiments, the centrifugation occurs at relative centrifugal force (RCF) of about 1300 g to about 1800 g. In some embodiments, the centrifugation occurs at relative centrifugal force (RCF) of about 1500 g. In some embodiments, the centrifugation occurs for about 1 minute to about 60 minutes. In some embodiments, the centrifugation occurs for about 10 minutes to about 30 minutes. In some embodiments, the centrifugation occurs for about 20 minutes.

In some embodiments, the platelets are at a concentration from about 1,000 platelets/μl to about 10,000,000 platelets/μl. In some embodiments, the platelets are at a concentration from about 50,000 platelets/μl to about 4,000,000 platelets/μl. In some embodiments, the platelets are at a concentration from about 100,000 platelets/μl to about 300,000,000 platelets/μl. In some embodiments, the platelets are at a concentration from about 1,000,000 to about 2,000,000. In some embodiments, the platelets are at a concentration of about 2,000,000 platelets/μl.

In some embodiments, the buffer is a loading buffer comprising the components as listed in Table 5 herein. In some embodiments, the loading buffer includes one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the loading buffer includes from about 0.5 mM to about 100 mM of the one or more salts. In some embodiments, the loading buffer includes from about 1 mM to about 100 mM (e.g., about 2 mM to about 90 mM, about 2 mM to about 6 mM, about 50 mM to about 100 mM, about 60 mM to about 90 mM, about 70 to about 85 mM) about of the one or more salts. In some embodiments, the loading buffer includes about 5 mM, about 75 mM, or about 80 mM of the one or more salts.

In some embodiments, the loading buffer includes one or more buffers, e.g., N-2-hydroxyethylpiperazine-N′-2-ethanesulfonic acid (HEPES), and/or sodium-bicarbonate (NaHCO₃). In some embodiments, the loading buffer includes from about 5 to about 100 mM of the one or more buffers. In some embodiments, the loading buffer includes from about 5 to about 50 mM (e.g., from about 5 mM to about 40 mM, from about 8 mM to about 30 mM, about 10 mM to about 25 mM) about of the one or more buffers. In some embodiments, the loading buffer includes about 10 mM, about 20 mM, about 25 mM, or about 30 mM of the one or more buffers.

In some embodiments, the loading buffer includes one or more saccharides, such as monosaccharides and disaccharides, including sucrose, maltose, trehalose, glucose, mannose, dextrose, and xylose. In some embodiments, the loading buffer includes from about 10 mM to about 1,000 mM of the one or more saccharides. In some embodiments, the loading buffer includes from about 50 to about 500 mM of the one or more saccharides. In embodiments, one or more saccharides is present in an amount of from 10 mM 10 to 500 mM. In some embodiments, one or more saccharides is present in an amount of from 50 mM to 200 mM. In embodiments, one or more saccharides is present in an amount from 100 mM to 150 mM.

In some embodiments, the loading buffer includes adding an organic solvent, such as ethanol, to the loading solution. In such a loading buffer, the solvent can range from about 0.1% (v/v) to about 5.0% (v/v), such as from about 0.3% (v/v) to about 3.0% (v/v), or from about 0.5% (v/v) to about 2% (v/v).

In some embodiments, the MRI agent includes one MRI agent. In some embodiments, the MRI agent includes one or more MRI agents.

In some embodiments, the methods further include incubating the MRI agent in the presence of the loading buffer prior to the treatment (e.g., contacting) step. In some embodiments, the methods further include incubating the loading buffer and a solution comprising the MRI agent and water at about 37° C. using different incubation periods. In some embodiments, the solution includes a concentration of about 0.1 nM to about 150 μM of the MRI agent. In some embodiments, the solution includes a concentration of about 1 nM to about 100 μM of the MRI agent. In some embodiments, the solution includes a concentration of about 10 nM to 50 μM of the MRI agent. In some embodiments, the solution includes a concentration of about 500 nM to about 50 μM of the MRI agent. In some embodiments, the solution includes a concentration of about 1 μM to about 30 μM of the MRI agent. In some embodiments, the solution includes a concentration of about 10 μM to about 30 μM of the MRI agent. In some embodiments, the incubation of the MRI agent in the presence of the loading buffer is performed from about 1 minute to about 2 hours. In some embodiments, the incubation is performed at an incubation period of from about 5 minutes to about 1 hour. In some embodiments, the incubation is performed at an incubation period of from about 10 minutes to about 30 minutes. In some embodiments, the incubation is performed at an incubation period of about 20 minutes.

In some embodiments, the methods further include incubating the MRI agent in the presence the loading buffer prior to the treatment (e.g., contacting) step.

In some embodiments, the methods further include mixing the platelets and the coupled cell penetrating peptide (CPP) and MRI agent (e.g., MRI agent-CPP) in the presence of the loading buffer at about room temperature (e.g., at about 20° C. to about 25° C.).

As used herein, “coupled,” describes attaching (e.g., complexing, conjugating) a cell penetrating peptide to an MRI agent. The coupling of the cell penetrating peptide and the MRI agent can be a covalent or a non-covalent coupling.

In some embodiments, the incubation is performed at an incubation period of from about 5 minutes to about 12 hours. In some embodiments, the incubation is performed at an incubation period of from about 10 minutes to about 6 hours. In some embodiments, the incubation is performed at an incubation period of from about 15 minutes to about 3 hours. In some embodiments, the incubation is performed at an incubation period of about 2 hours. In some embodiments, the final product includes platelets and the MRI-agent CPP at a volume ratio of 10:1, with a range in volume ratio of about 1 to about 50.

In some embodiments, the concentration of MRI agent in the MRI agent-loaded platelets is from about 0.1 nM to about 10 μM. In some embodiments, the concentration of MRI agent in the MRI agent-loaded platelets is from about 1 nM to about 1 μM. In some embodiments, the concentration of MRI agent in the MRI agent-loaded platelets is from about 10 nM to 10 μM. In some embodiments, the concentration of MRI agent in the MRI-loaded platelets is about 100 nM.

In some embodiments, the methods further include drying the MRI agent-loaded platelets. In some embodiments, the drying step includes freeze-drying the MRI agent-loaded platelets. In some embodiments, the methods further include rehydrating the MRI agent-loaded platelets obtained from the drying step.

In some embodiments, MRI agent-loaded platelets are prepared by using any of the variety of methods provided herein.

In some embodiments, rehydrated MRI agent-loaded platelets are prepared by any one method comprising rehydrating the MRI agent-loaded platelets provided herein.

The MRI agent-loaded platelets may be used, for example, in therapeutic applications as disclosed herein. Additionally or alternatively, the MRI agent-loaded platelets may be employed in functional assays. In some embodiments, the MRI agent-loaded platelets are cold stored, cryopreserved, or lyophilized (to produce thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table A. Additional exemplary lyophilization methods can be found in U.S. Pat. Nos. 7,811,558, 8,486,617, and 8,097,403. An exemplary spray-drying method includes: combining nitrogen, as a drying gas, with a loading buffer according to the present disclosure, then introducing the mixture into GEA Mobile Minor spray dryer from GEA Processing Engineering, Inc. (Columbia Md., USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of10 to 35 minutes. The final step in spray drying is preferentially collecting the dried mixture. The dried composition in some embodiments is stable for at least six months at temperatures that range from −20° C. or lower to 90° C. or higher.

TABLE A
Exemplary Lyophilization Protocol

| | Step | Temp. Set | | Type | Duration | | Pressure Set |
|---|---|---|---|---|---|---|---|
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | Var | | N/A |
| Primary Dry | P1 | −40° | | Hold | 1.5 | Hrs | 0 mT |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | S1 | 27° | C. | Hold | >8 | Hrs | 0 mT |

In some embodiments, the step of drying the MRI agent-loaded platelets that are obtained as disclosed herein, such as the step of freeze-drying the MRI agent-loaded platelets that are obtained as disclosed herein, includes incubating the platelets with a lyophilizing agent. In some embodiments, the lyophilizing agent is polysucrose. In some embodiments, the lyophilizing agent is a non-reducing disaccharide. Accordingly, in some embodiments, the methods for preparing MRI agent-loaded platelets further include incubating the MRI agent-loaded platelets with a lyophilizing agent. In some embodiments, the lyophilizing agent is a saccharide. In some embodiments, the saccharide is a disaccharide, such as a non-reducing disaccharide.

In some embodiments, the platelets are incubated with a lyophilizing agent for a sufficient amount of time and at a suitable temperature to load the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrrolidone (PVP), starch, and hydroxyethyl starch (HES). In some embodiments, exemplary lyophilizing agents can include a high molecular weight polymer, into the loading composition. By “high molecular weight” it is meant a polymer having an average molecular weight of about or above 70 kDa. Non-limiting examples are polymers of sucrose and epichlorohydrin. In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%.

In some embodiments, the process for preparing a composition includes adding an organic solvent, such as ethanol, to the loading solution. In such a loading solution, the solvent can range from 0.1% to 5.0% (v/v).

Within the process provided herein for making the compositions provided herein, addition of the lyophilizing agent can be the last step prior to drying. However, in some embodiments, the lyophilizing agent is added at the same time or before the MRI agent, the cryoprotectant, or other components of the loading composition. In some embodiments, the lyophilizing agent is added to the loading solution, thoroughly mixed to form a drying solution, dispensed into a drying vessel (e.g., a glass or plastic serum vial, a lyophilization bag), and subjected to conditions that allow for drying of the solution to form a dried composition.

An exemplary saccharide for use in the compositions disclosed herein is trehalose. Regardless of the identity of the saccharide, it can be present in the composition in any suitable amount. For example, it can be present in an amount of 1 mM to 1 M. In embodiments, it is present in an amount of from 10 mM 10 to 500 mM. In some embodiments, it is present in an amount of from 20 mM to 200 mM. In some embodiments, it is present in an amount from 40 mM to 100 mM. In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

The step of incubating the platelets to load them with a cryoprotectant or as a lyophilizing agent includes incubating the platelets for a time suitable for loading, as long as the time, taken in conjunction with the temperature, is sufficient for the cryoprotectant or lyophilizing agent to come into contact with the platelets and, preferably, be incorporated, at least to some extent, into the platelets. In embodiments, incubation is carried out for about 1 minute to about 180 minutes or longer.

The step of incubating the platelets to load them with a cryoprotectant or lyophilizing agent includes incubating the platelets and the cryoprotectant at a temperature that, when selected in conjunction with the amount of time allotted for loading, is suitable for loading. In general, the composition is incubated at a temperature above freezing for at least a sufficient time for the cryoprotectant or lyophilizing agent to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

In various embodiments, the bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SA/V ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6 cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SA/V ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, the lyophilizing agent as disclosed herein may be a high molecular weight polymer. By “high molecular weight” it is meant a polymer having an average molecular weight of about or above 70 kDa and up to 1,000,000 kDa Non-limiting examples are polymers of sucrose and epichlorohydrin (polysucrose). Although any amount of high molecular weight polymer can be used, it is preferred that an amount be used that achieves a final concentration of about 3% to 10% (w/v), such as 3% to 7%, for example 6%. Other non-limiting examples of lyoprotectants are serum albumin, dextran, polyvinyl pyrrolidone (PVP), starch, and hydroxyethyl starch (HES).

In some embodiments, the loading buffer includes an organic solvent, such as an alcohol (e.g., ethanol). In such a loading buffer, the amount of solvent can range from 0.1% to 5.0% (v/v).

In some embodiments, the MRI agent-loaded platelets prepared as disclosed herein have a storage stability that is at least about equal to that of the platelets prior to the loading of the MRI agent.

The loading buffer may be any buffer that is non-toxic to the platelets and provides adequate buffering capacity to the solution at the temperatures at which the solution will be exposed during the process provided herein. Thus, the buffer may include any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TB S). Likewise, it may include one or more of the following buffers: propane-1,2,3-tricarboxylic (tricarballylic); benzenepentacarboxylic; maleic; 2,2-dimethyl succinic; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES).

Flow cytometry can be used to obtain a relative quantification of loading efficiency by measuring the mean fluorescence intensity of the MRI agent in the MRI agent-loaded platelets. Platelets can be evaluated for functionality by adenosine diphosphate (ADP), collagen, arachidonic acid, thrombin receptor activating peptide (TRAP), and/or any other platelet agonist known in the art for stimulation post-loading.

In some embodiments, the MRI agent-loaded platelets are lyophilized. In some embodiments, the MRI agent-loaded platelets are cryopreserved.

In some embodiments, the MRI agent-loaded platelets retain the loaded MM agent upon rehydration and release the MRI agent upon stimulation by endogenous platelet activators.

In some embodiments, the dried platelets (such as freeze-dried platelets) retain the loaded MRI agent upon rehydration and release the MRI agent upon stimulation by endogenous platelet activators. In some embodiments, at least about 10%, such as at least about 20%, such as at least about 30% of the MRI agent is retained. In some embodiments, from about 10% to about 20%, such as from about 20% to about 30% of the MRI agent is retained.

Any suitable MRI agent can be loaded in a platelet. For example, any agent suitable for magnetic resonance imaging can be loaded into a platelet.

In some embodiments, the MRI agent can include Gadolinium. In some embodiments, Gadolinium (Gd (III)) can be coupled with a chelator. In some embodiments, the chelator can be DOTA (tetrxetan). In some embodiments, the MRI agent can be a complex of Gd and DOTA (e.g., Gd-DOTA).

In some embodiments, the MRI agent can be a nanoparticle. Any suitable nanoparticle (e.g., magnetic nanoparticle) can be loaded into the platelet. In some embodiments, the nanoparticle is a FeO₃ nanoparticle.

In some embodiments, the nanoparticle can be about 15 nm to about 100 nm in diameter. In some embodiments, the nanoparticle can be about 20 nm to about 90 nm in diameter. In some embodiments, the nanoparticle can be about 30 nm to about 80 nm in diameter. In some embodiments, the nanoparticle can be about 40 nm to about 70 nm in diameter. In some embodiments, the nanoparticle can be about 50 nm to about 60 nm in diameter. In some embodiments, the nanoparticle can be about 20 nm to about 30 nm in diameter.

In some embodiments, the nanoparticles can be at a concentration of about 1×10⁻²⁰ to about 1×10¹⁴ particles/mL. In some embodiments, the nanoparticles can be at a concentration of about 1×10¹⁹ to about 1×10¹⁵ particles/mL. In some embodiments, the nanoparticles can be at a concentration of about 1×10⁻¹⁸ to about 1×10⁻¹⁶ particles/mL.

In some embodiments, the MRI agent loaded platelets can be coupled (e.g., conjugated) with a cell penetrating peptide. Cell penetrating peptides are peptides that can facilitate cellular uptake of various cargo (e.g., nucleic acid, protein, metabolites, lipids, nanoparticles, metals, etc.). Generally, cell penetrating peptides can cross a cellular membrane by direct penetration in the membrane, endocytosis mediated entry, or translocation through the formation of a transitory structure, although additional mechanisms are known.

The HIV Tat protein is an example of a cell penetrating peptide. The Tat protein includes between 86 and 101 amino acids depending on the subtype. Tat is a regulatory protein that enhances the viral transcription efficiency. Tat also contains a protein transduction domain which functions as a cell-penetrating domain allowing Tat to cross cellular membranes.

In some embodiments, an MRI agent can be coupled with HIV Tat protein. In some embodiments, an MRI agent can be coupled with a portion of the HIV Tat protein. In some embodiments, an MRI agent can be coupled with a portion of the Tat protein: L-Tat₄₉₋₅₇ as described in Mishra, R., et. al., Cell-Penetrating Peptides and Peptide Nucleic Acid-Coupled Mill Contrast Agents: Evaluation of Cellular Delivery and Target Binding, Bioconjugate Chem. 20, 1860-1868 (2009)).

In some embodiments, the MRI agent can be coupled with a lipophilic moiety. Some non-limiting examples include a lipid coated nanoparticle or a liposome.

In some embodiments, the MRI agent can be coupled with a cyclodextrin cage.

In some embodiments, the one or more other components that are loaded in the platelets include Prostaglandin E1.

In some embodiments, the one or more other components that are loaded in the platelets do not include Prostaglandin E1.

In some embodiments, the one or more other components that are loaded in the platelets include a glycoprotein IIb/IIIa inhibitor (GP IIb/IIIa). Non-limiting examples of GP IIb/IIIa inhibitors include GR144053, eptifibatide, ethylenediaminetetraacetic acid (EDTA), abciximab, tirofiban.

In some embodiments, the one or more other components that are loaded in the platelets include GR144053.

In some embodiments, the one or more other components that are loaded in the platelets do not include GR144053.

In some embodiments, the one or more other components that are loaded in the platelets include eptifibatide.

In some embodiments, the one or more other components that are loaded in the platelets do not include eptifibatide.

Exemplary protocols that employ the foregoing agents or procedures are shown below:

### Cell Penetrating Peptide

### Background

Cell penetrating peptides are peptides that can facilitate cellular uptake of various cargo (e.g., nucleic acid, protein, metabolites, lipids, nanoparticles, metals, etc.). Cargo can be coupled (e.g., conjugated) to a cell penetrating peptide either covalently or non-covalently. A cell penetrating peptide conjugated to cargo can transport the cargo across a cellular membrane, generally via endocytosis, however other mechanisms are known in the art.

### Protocol

As described here and in the Examples below, prepare the MRI agent (e.g., FITC-CPP-Ga-DOTA or FITC-labeled nanoparticles) in aqueous buffer at room temperature. Incubate the FITC-CPP-Ga-DOTA or FITC-labeled nanoparticles with platelets up to 3 hours at 37° C. on a rocker with low frequency agitation. Transfected platelets may be lyophilized to create Thrombosomes with an MRI agent. Fluorescently labeled FITC-CPP-Ga-DOTA or FITC-labeled nanoparticles can be detected via flow cytometry and visualized using fluorescence microscopy.

In some embodiments, MRI agent-loaded platelets, MRI agent-loaded platelet derivatives, or MRI agent-loaded thrombosomes may shield the MRI agent from exposure in circulation, thereby reducing or eliminating systemic toxicity (e.g. cardiotoxicity) associated with the MRI agent. In some embodiments, MRI agent-loaded platelets, MRI agent-loaded platelet derivatives, or MRI agent-loaded thrombosomes may also protect the MRI agent from metabolic degradation or inactivation. In some embodiments, MRI agent delivery with MRI agent-loaded platelets, MRI agent-loaded platelet derivatives, or MRI agent-loaded thrombosomes may therefore be advantageous in treatment of diseases such as cancer, since MRI agent-loaded platelets, MRI agent-loaded platelet derivatives, or MRI agent-loaded thrombosomes facilitate targeting of cancer cells while mitigating systemic side effects. In some embodiments, MRI agent-loaded platelets, MRI agent-loaded platelet derivatives, or MRI agent-loaded thrombosomes may be used in any therapeutic setting in which expedited healing process is required or advantageous.

In some embodiments, provided herein is a method of enhancing diagnosis and treatment of a disease as disclosed herein, comprising administering MRI agent-loaded platelets, MRI agent-loaded platelet derivatives, or MRI agent-loaded thrombosomes as disclosed herein. In some embodiments, provided herein is a method of treating a disease as disclosed herein, comprising administering cold stored, room temperature stored, cryopreserved thawed, rehydrated, and/or lyophilized platelets, platelet derivatives, or thrombosomes as disclosed herein. In some embodiments, the disease is cancer. In some embodiments, the disease is Traumatic Brain injury. In some embodiments, the disease is cancer. In some embodiments, the disease is Traumatic Brain injury. In some embodiments, the disease is stroke. In some embodiments, the disease is an embolism. In some embodiments, the disease is a hemorrhage.

Examples of diseases (therapeutic indications) the MRI agent-loaded platelets may enhance diagnosis and treatment with include, in a non-limiting way:

Therapeutic indications

Acute lymphoblastic leukemia (ALL)
Acute myeloid leukemia (AML)
Breast cancer (can also be used as an adjuvant
therapy for metastasized breast cancer post-surgery)
Gastric cancer
Hodgkin lymphoma
Neuroblastoma
Non - Hodgkin lymphoma
Ovarian cancer
Cervical cancer
Small cell lung cancer
Non-small cell lung cancer (NSCLC)
Soft tissue and bone sarcomas
Thyroid cancer
Transitional cell bladder cancer
Wilms tumor
Neuroendocrine tumors
Pancreatic cancer
Multiple myeloma
Renal cancer
Glioblastoma
Prostate cancer
Sarcoma
Colon cancer
Melanoma
Colitis
Chronic inflammatory demyelinating polyneuropathy
Guillain - Barre syndrome
Immune Thrombocytopenia
Kawasaki disease
Lupus
Multiple Sclerosis
Myasthenia gravis
Myositis
Cirrhosis with refractory ascites
Hepatorenal syndrome (used in combination with
vasoconstrictive drugs)
Nephrotic syndrome (for patient with albumin <2
g/dL with hypovolaemia and/or pulmonary edema)
Organ transplantation
Paracentesis
Hypovolemia
Aneurysms
Artherosclerosis
Cancer
Cardiovascular diseases (post - myocardial infarction
remodeling, cardiac regeneration, cardiac fibrosis,
viral myocarditis, cardiac hypertrophy, pathological
cardiac remodeling)
Genetic disorders
Infectious diseases
Metabolic diseases
Neoangiogenesis
Opthalmic conditions (retinal angiogenesis, ocular
hypertension, glaucoma, diabetic macular edema,
diabetic retinopathy, macular degeneration)
Hypercholesterolemia
Pulmonary hypertension

Examples of MRI agent and therapeutic indications for MRI agent(s) to be loaded into platelets are as follows:

| MRI Agent | Therapeutic indications |
|---|---|
| | Aneurysms |
| | Artherosclerosis |
| | Cancer |
| | Cardiovascular diseases (post - myocardial infarction |
| | remodeling, cardiac regeneration, cardiac fibrosis, |
| | viral myocarditis, cardiac hypertrophy, pathological |
| | cardiac remodeling) |
| | Genetic disorders |
| | Metabolic diseases |
| | Neoangiogenesis |
| | Opthalmic conditions (retinal angiogenesis) |
| | Pulmonary hypertension |

While the embodiments of the invention are amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### EXAMPLE 1

### MRI-Agent Loaded Platelets

Protocol 1: Loading Platelets with an MRI Agent

Acidify the platelet pool to pH 6.6-6.8 using 4 μL 1M Acid Citrate Dextrose solution per 1 mL pooled platelet rich plasma.

Obtain platelet count in solution using Coulter AcT Diff hematology analyzer.

Isolate platelets via centrifugation at 845×g for 10 minutes at room temperature, with gentle acceleration and braking.

Prepare incubation solutions of FITC-CPP (FITC-TAT) or FITC-labeled magnetic nanoparticles.

Re-suspend platelets in loading buffer (Table 1) or desired incubation solution at a concentration of 500,000 platelets/μL and incubate at 37° C. with low frequency agitation on a rocker for up to 3 hours.

Wash platelets with loading buffer (Table 1) 3× to remove unloaded agents (e.g., FITC-TAT, FITC-labeled magnetic nanoparticles) and use the remaining sample for plate reader, flow cytometer, and/or microscope analysis.

Platelets are isolated and pooled by centrifugation to any concentration the isolation technology can achieve. The typical concentration is 5×10⁶ platelets/μl. The platelet medium can be altered to change the proportion of excipients, or to exchange excipients for similar products. The platelet medium is then replaced with a buffer composed of:

TABLE 1
Loading Buffer:

| | Concentration |
|---|---|
| | (mM, except where |
| Component | otherwise indicated) |
| NaCl | 750 |
| KCl | 48 |
| HEPES | 95 |
| NaHCO₃ | 120 |
| Dextrose | 3 |
| Trehalose | 0.1M |
| Ethanol | 1.00% (v/v) |
| Dansyl-EACA/ESCA (1:1000) | 0, 50, or 100 |

After resuspension the platelets were incubated with either 5-100 μM CPP (L-TAT 49-57, See Mishra., R., (2009)) conjugated to FITC and Gd-DOTA or FeO₃ nanoparticles at about between 1×10¹⁹ and about 1×10¹⁵ nanoparticles/mL buffer (average particle diameter of about 20-30 nm, labeled with FITC) in the loading buffer for up to 4 hours at 37° C. The loaded platelets are then used for applications which include, but are not limited to, cryopreservation, lyopreservation, and immediate use in therapeutic functional or diagnostic assays.

The results for the above formulation are provided herein. A Tecan Infinite M200 Pro plate reader was used for quantification of MRI agent loading. A Novocyte flow cytometer was used to determine both MRI agent loading and percent of platelets effectively loaded. An Olympus microscope was used to visualize the loading into platelets.

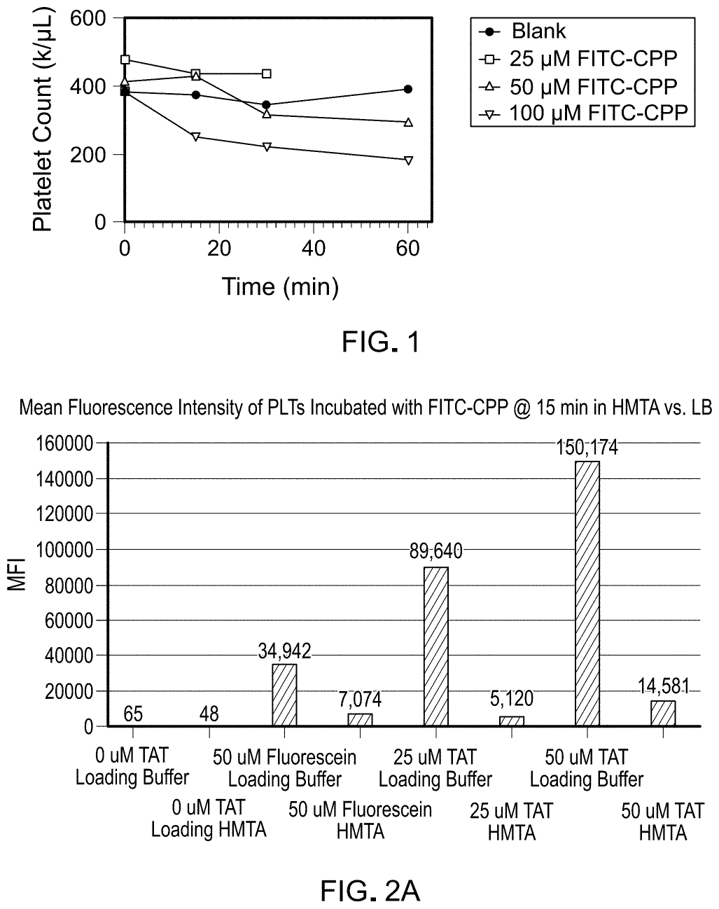

FIG. 1 shows pooled apheresis platelets incubated with FITC-labeled TAT peptide in loading buffer. The platelets were incubated for 15 to 60 minutes at 37° C. with low frequency agitation on a rocker. After incubation, the platelet counts were analyzed on an AcT-Diff hemacytometer. The data show that platelet counts are stable over time up to a concentration of 50 μM FITC-TAT.

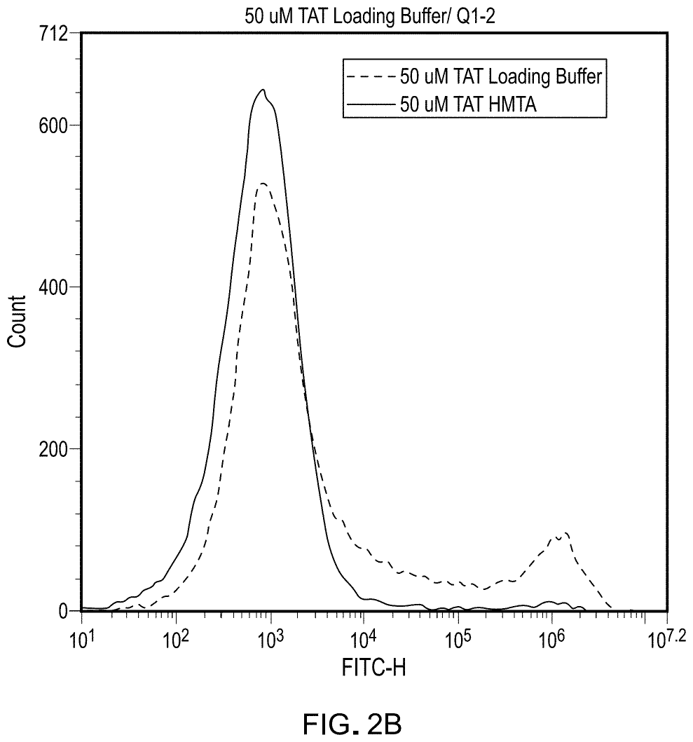

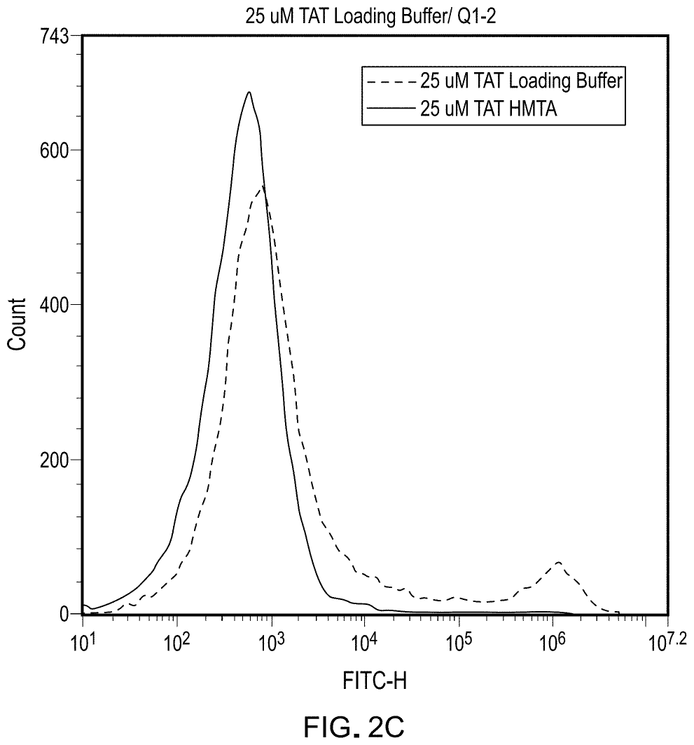

Pooled apheresis platelets were incubated with FITC-labeled TAT peptide in either HMTA or loading buffer. Platelets are incubated for 15 minutes at 37° C. with low frequency agitation on a rocker. After washing, the platelets were analyzed by flow cytometry for FITC-TAT loading. FIG. 2A shows the mean fluorescence values for each sample. FIGS. 2B and 2C are histograms are for 50 μM (FIG. 2B) or 25 μM (FIG. 2C) FITC-TAT concentrations during loading, respectively. Negative controls included 0 μM FITC-TAT in both loading buffer (Table 1) and HMTA and 50 μM fluorescein in both loading buffer and HMTA.

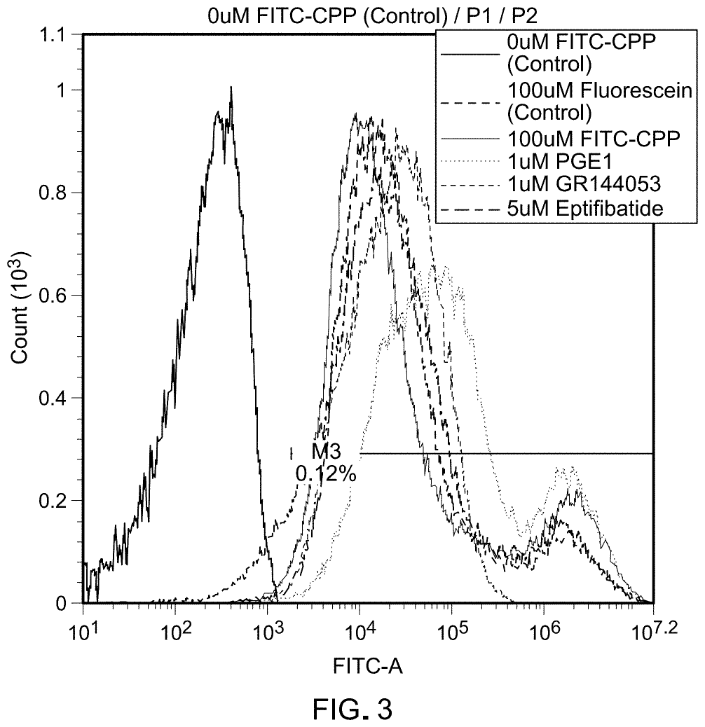

FIG. 3 shows a flow cytometry histograms of samples incubated with 100 μM FITC-CPP for 60 minutes in the presence of platelet anti-aggregation compounds, including PGE1, GR144053, and eptifibatide. Negative controls included 0 μM FITC-TAT in loading buffer and 50 μM fluorescein in loading buffer. All samples were incubated in loading buffer at 37° C. with low frequency agitation on a rocker.

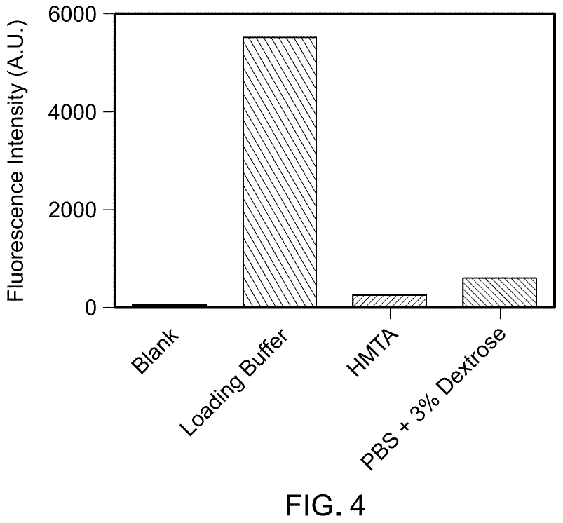

FIG. 4 shows the effect of different buffers on FITC-TAT loading into platelets as measured by fluorescence intensity. Minimal fluorescence was detected with HMTA buffer and PBS with 3% dextrose.

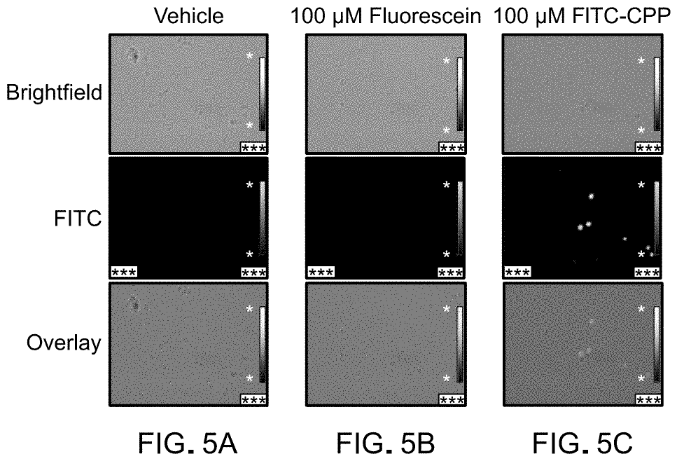

FIGS. 5A-C shows brightfield, FITC, and overlaid images of 0 μM FITC-TAT (“Vehicle”) (FIG. 5A), 100 μM fluorescein (FIG. 5B), and 100 μM FITC-TAT (“FITC-CPP”) (FIG. 5C) incubated for 30 minutes at 37° C. with low frequency agitation on a rocker in loading buffer containing the additive indicated herein. Each image is representative. The scale bars are 10 μm. The overlay image in the bottom right corner shows FITC-TAT co-localizing with platelets.

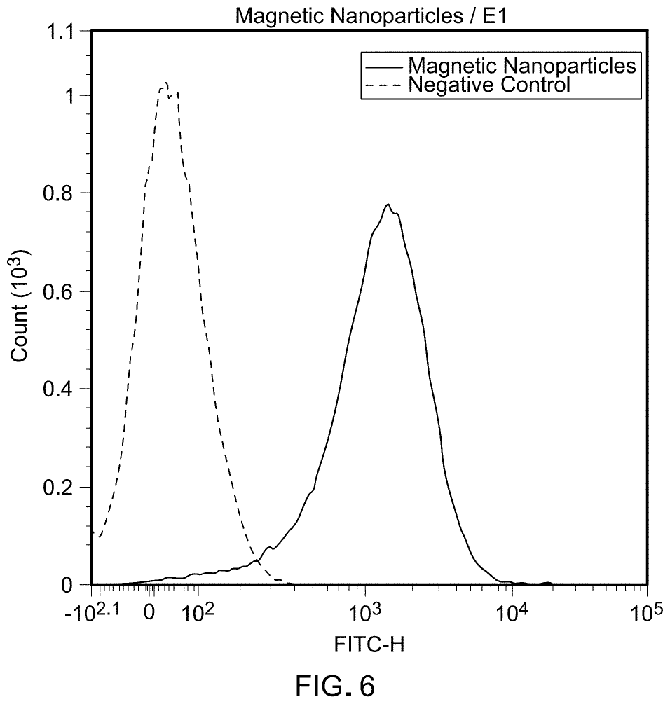

Flow cytometry histograms of samples incubated with either loading buffer or a solution of magnetic nanoparticles (size range between 20-30 nm) labeled with FITC. The data show that platelets are capable of endocytosing magnetic nanoparticles (FIG. 6). All samples were incubated in loading buffer at 37° C. with low frequency agitation on a rocker.

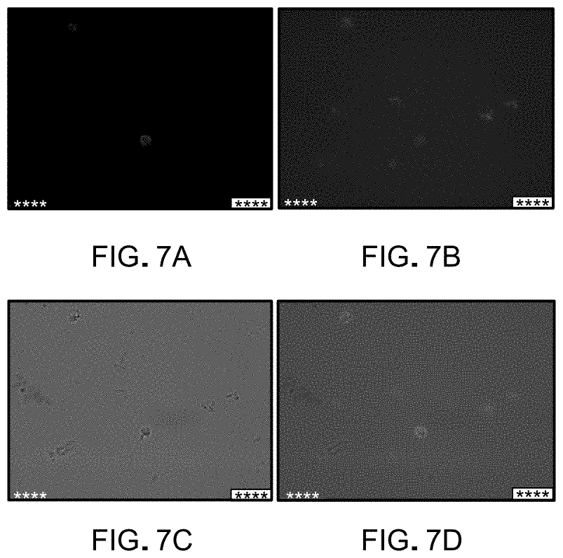

FIGS. 7A-D shows Texas Red (FIG. 7A), brightfield (FIG. 7C), FITC conjugated nanoparticles (FIG. 7B), and overlaid (FIG. 7D) images of samples incubated with nanoparticles (size range between 20-30 nm) labeled with FITC-TAT for 3 hours at 37° C. with low frequency agitation on a rocker in loading buffer. Each image is representative. The scale bars are 10 μm.

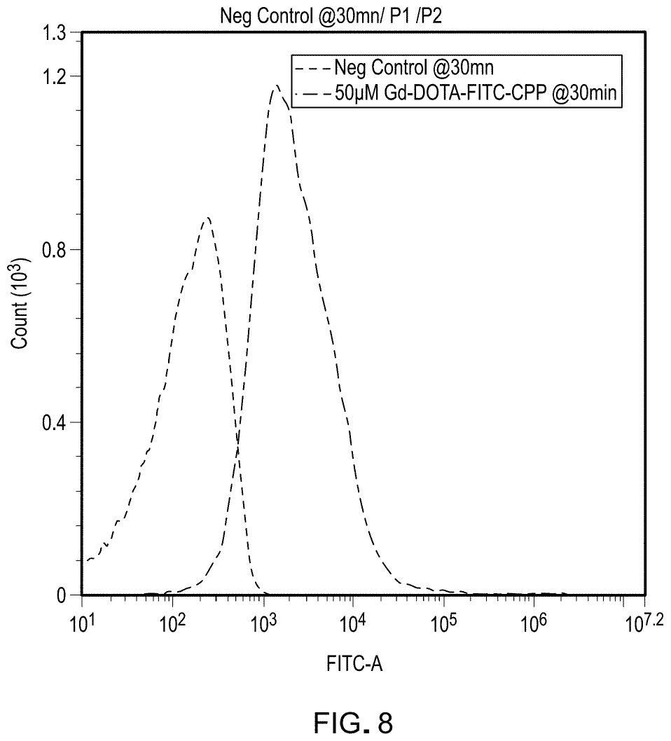

FIG. 8 shows flow cytometry histograms of platelet samples incubated with either loading buffer (left peak) or a 50 μM FITC-CPP-Gd-DOTA solution (right peak) for 30 minutes. The data show that 76% of flow events showed fluorescent signal above background. All platelet samples were incubated in loading buffer at 37° C. with low frequency agitation on a rocker.

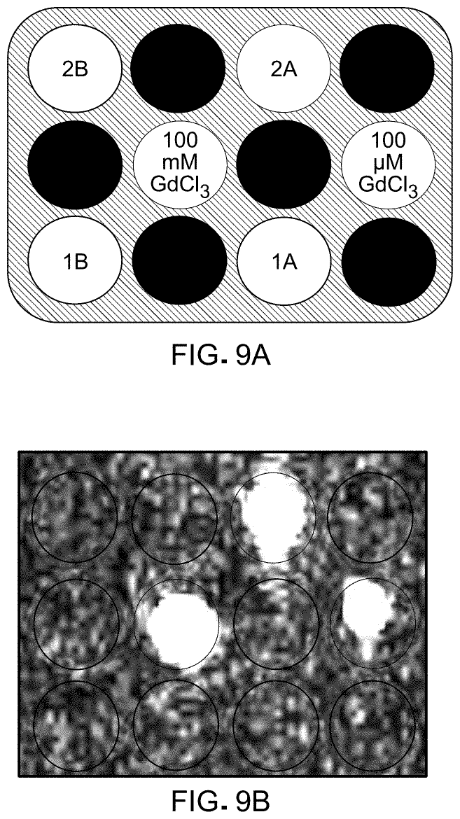

FIGS. 9A-B show a schematic (FIG. 9A) and magnetic resonance imaging (FIG. 9B). As shown in FIG. 9A, samples 1A, 1B, and 2B are negative controls, sample 2A includes Gd-DOTA-FITC-CPP with platelets (400 K/μL), samples indicated with either 100 mM or 100 μM GdCl₃ are positive controls. Sample 1A included loading buffer and platelets (400 K/μL), sample 1B included loading buffer alone, sample 2A included loading buffer, Gd-DOTA-CPP-FITC, and platelets (400 K/μL), and sample 2B included loading buffer and Gd-DOTA-FITC-CPP only (washed) as another negative control. The magnetic resonance imaging data shown in FIG. 9B shows detection in the two positive control samples and also sample 2A which included loading buffer, Gd-DOTA-CPP-FITC, and platelets (400 K/μL), thus showing loading of Gd-DOTA-CPP-FITC into platelets.

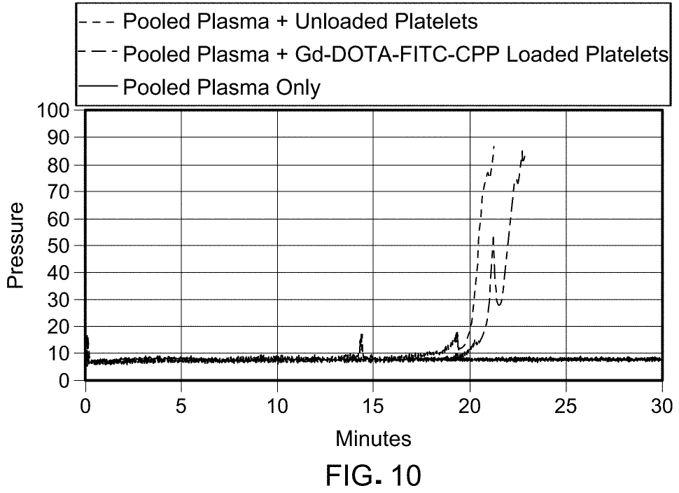

FIG. 10 is a graph showing post-cryopreservation occlusion time of platelets loaded with Gd-DOTA-FITC-CPP with plasma only (negative control) (shown as bottom line), pooled, unloaded platelets (positive control), and Gd-DOTA-FITC-CPP loaded platelets. The occlusion times were as follows: negative control=0 minutes, positive control (21 minutes), and Gd-DOTA-FITC-CPP loaded platelets (22 minutes). Thus, Gd-DOTA-FITC-CPP loaded platelets had a similar occlusion time to unloaded platelets indicating that such loaded platelets retain hemostatic function.

### Exemplary Embodiments

Embodiment 1 is a method of preparing MRI agent-loaded platelets, comprising:
treating platelets with an MRI agent coupled to a cell penetrating peptide;
and a loading buffer comprising a salt, a base, a loading agent, and optionally at least one organic solvent,
   to form the MRI agent-loaded platelets.

Embodiment 2 is a method of preparing MRI agent-loaded platelets, comprising:
a) providing platelets; and
b) treating the platelets with an MRI agent coupled to a cell penetrating peptide; and
a loading buffer comprising a salt, a base, a loading agent, and optionally at least one organic solvent
   to form the MRI agent-loaded platelets.

Embodiment 3 is the method of any one of the preceding embodiments, wherein the platelets are treated with the MRI agent coupled to a cell penetrating peptide and with the loading buffer sequentially, in either order.

Embodiment 4 is method of preparing MRI agent-loaded platelets, comprising:
A) treating the platelets with a loading buffer comprising a salt, a base, a loading agent, and optionally at least one organic solvent to form a first composition; and
B) treating the first composition with an MRI agent coupled to a cell penetrating peptide, to form the MRI agent-loaded platelets.

Embodiment 5 is the method of embodiment 1 or 2, wherein the platelets are treated with the MRI agent coupled to the cell penetrating peptide and with the loading buffer concurrently.

Embodiment 6 is a method of preparing MRI agent-loaded platelets, comprising:
treating the platelets with an MRI agent in the presence of a cell penetrating peptide and a loading buffer comprising a salt, a base, a loading agent, and optionally at least one organic solvent to form the MRI agent-loaded platelets.

Embodiment 7 is the method of any one of the preceding embodiments, wherein the platelets are pooled from a plurality of donors.

Embodiment 8 is a method of preparing MRI agent-loaded platelets comprising
A) pooling platelets from a plurality of donors; and
B) treating the platelets from step (A) with an MRI agent coupled to a cell penetrating peptide; and with a loading buffer comprising a salt, a base, a loading agent, and optionally at least one organic solvent, to form the MRI agent-loaded platelets.

Embodiment 9 is a method of preparing MRI agent-loaded platelets comprising
A) pooling platelets from a plurality of donors; and
B)
   a. treating the platelets from step (A) with an MRI agent coupled to a cell penetrating peptide to form a first composition; and
   b. treating the first composition with a loading buffer comprising a salt, a base, a loading agent, and optionally at least one organic solvent, to form the MRI agent-loaded platelets.

Embodiment 10 A method of preparing MRI agent-loaded platelets comprising
pooling platelets from a plurality of donors; and treating the platelets from step (A) with a loading buffer comprising a salt, a base, a loading agent, and optionally at least one organic solvent, to form a first composition; and
treating the first composition with an MRI agent coupled to a cell penetrating peptide to form the MRI agent-loaded platelets.

Embodiment 11 is a method of preparing MRI agent-loaded platelets comprising
pooling platelets from a plurality of donors; and
treating the platelets with an MRI agent coupled to a cell penetrating peptide and a loading buffer comprising a salt, a base, a loading agent, and optionally at least one organic solvent, to form the MRI agent-loaded platelets.

Embodiment 12 is the method of any one of the preceding embodiments, wherein the loading buffer comprises optionally at least one organic solvent.

Embodiment 13 is the method of any one of the preceding embodiments, wherein the loading agent is a monosaccharide or a disaccharide.

Embodiment 14 is the method of any one of the preceding embodiments, wherein the loading agent is sucrose, maltose, dextrose, trehalose, glucose, mannose, or xylose.

Embodiment 15 is the method of any one of the preceding embodiments, wherein the platelets are isolated prior to a treating step.

Embodiment 16 is the method of any one of the preceding embodiments, wherein the platelets are selected from the group consisting of fresh platelets, stored platelet, and any combination thereof.

Embodiment 17 is the method of any one of the preceding embodiments, wherein the MRI agent comprises Gadolinium.

Embodiment 18 is the method of any one of the preceding embodiments, wherein the MRI agent comprises a nanoparticle.

Embodiment 19 is the method of any one of the preceding embodiments, wherein the cell penetrating peptide is Tat, or a portion thereof.

Embodiment 20 is the method of any one of the preceding embodiments, wherein the platelets are loaded with the MRI agent in a period of time of 1 minute to 48 hours.

Embodiment 21 is the method of any one of the preceding embodiments, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

Embodiment 22 is the method of any one of the preceding embodiments, further comprising cold storing, cryopreserving, freeze-drying, thawing, rehydrating, and combinations thereof the MRI agent-loaded platelets.

Embodiment 23 The method of embodiment 22, wherein the drying step comprises freeze-drying the MRI agent-loaded platelets.

Embodiment 24 is the method of embodiment 22 or 23, further comprising rehydrating the MRI agent-loaded platelets obtained from the drying step.

Embodiment 25 are MRI agent-loaded platelets prepared by the method of any one of the preceding embodiments.

Embodiment 26 are MRI agent-loaded platelets prepared by a method comprising rehydrating the MRI agent-loaded platelets of embodiment 25.

Embodiment 27 is the method of any one of the preceding embodiments, wherein the method does not comprise treating the platelets with an organic solvent.

Embodiment 28 is the method of any one of embodiments 4, 9, or 10, wherein the method does not comprise treating the first composition with an organic solvent.

Embodiment 29 is the method of any one of the preceding embodiments, wherein the method comprises treating the platelets with Prostaglandin E1.

Embodiment 30 is the method of any one of the preceding embodiments, wherein the method does not comprise treating the platelets with Prostaglandin E1.

Embodiment 31 is the method of any one of the preceding embodiments, wherein the method comprises treating the plates with GR144053.

Embodiment 32 is the method of any one of the preceding embodiments, wherein the method does not comprise treating the platelets with GR144053.

Embodiment 33 is the method of any one of the preceding embodiments, wherein the method comprises treating the platelets with eptifibatide.

Embodiment 34 is the method of any one of the preceding embodiments, wherein the method does not comprise treating the platelets with eptifibatide.

1. A method of preparing MRI agent-loaded platelets, comprising:
contacting platelets with an MRI agent coupled to a cell penetrating peptide,
and a loading buffer comprising a salt, a base, a loading agent, and optionally at least one organic solvent,
to form the MRI agent-loaded platelets.

2. A method of preparing MRI agent-loaded platelets, comprising:
(a) providing platelets; and
(b) contacting the platelets with an MRI agent coupled to a cell penetrating peptide; and a loading buffer comprising a salt, a base, a loading agent, and optionally at least one organic solvent
to form the MRI agent-loaded platelets.

3. The method of claim 1, wherein the platelets are treated with the MRI agent coupled to a cell penetrating peptide and with the loading buffer sequentially, in either order, or concurrently.

4. The method of claim 1, wherein the platelets are pooled from a plurality of donors.

5. A method of preparing MRI agent-loaded platelets comprising
A) pooling platelets from a plurality of donors; and
B) contacting the platelets from step (A) with an MRI agent coupled to a cell penetrating peptide; and with a loading buffer comprising a salt, a base, a loading agent, and optionally at least one organic solvent, to form the MRI agent-loaded platelets.

6. The method of claim 1, wherein the loading buffer comprises at least one organic solvent.

7. The method of claim 1, wherein the loading agent is a monosaccharide or a disaccharide.

8. The method of claim 1, wherein the loading agent is sucrose, maltose, dextrose, trehalose, glucose, mannose, or xylose.

9. The method of claim 1, wherein the platelets are isolated prior to a contacting step.

10. The method of claim 1, wherein the platelets are selected from the group consisting of fresh platelets, stored platelet, and any combination thereof.

11. The method of claim 1, wherein the MRI agent comprises Gadolinium or a nanoparticle.

12. The method of claim 1, wherein the cell penetrating peptide is Tat, or a portion thereof.

13. The method of claim 1, wherein the platelets are loaded with the MRI agent in a period of time of 1 minute to 48 hours.

14. The method of claim 1, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

15. The method of claim 1, further comprising cold storing, cryopreserving, freeze-drying, thawing, rehydrating, or combinations thereof the MRI agent-loaded platelets.

16. The method of claim 15, wherein the method comprises freeze-drying the MRI agent-loaded platelets.

17. The method of claim 16, further comprising rehydrating the MRI agent-loaded platelets obtained from the freeze-drying step.

18. MRI agent-loaded platelets prepared by the method of claim 1.

19. MRI agent-loaded platelets prepared by a method comprising rehydrating the MRI agent-loaded platelets of claim 18.

20. The method of claim 1, wherein the method does not comprise contacting the platelets with an organic solvent.

21. The method of claim 1, wherein the method comprises optionally contacting the platelets with Prostaglandin E1.

22. The method of claim 1, wherein the method comprises optionally contacting the plates with a glycoprotein IIb/IIIa inhibitor.
